(19) United States
(12) Patent Application Publication
(10) Pub. No.: US 2010/0297610 A1
(43) Pub. Date: Nov. 25, 2010

(54) MOLECULARLY IMPRINTED POLYMERS FOR DETECTING HIV-1

(75) Inventor: Robert L. Jones, Columbia, MD (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Columbia BioSystems, Baltimore, MD (US)

(21) Appl. No.: 12/761,013

(22) Filed: Apr. 15, 2010

Related U.S. Application Data
(60) Provisional application No. 61/169,450, filed on Apr. 15, 2009.
(60) Provisional application No. 61/222,165, filed on Jul. 1, 2009.

(51) Int. Cl. C12Q 1/70; C08G 73/06

(52) U.S. Cl. 435/5; 528/423

(57) ABSTRACT

The invention described herein provides molecularly imprinted polymers (MIPs) that are capable of binding to virus, and methods for detecting and/or identifying specific virus particles utilizing Molecularly Imprinted Polymers (MIPs). The virus particles of the invention include HIV-1, HIV-2, HTLV-1, HTLV-2, HPV, HBV, and HCV. The methods of the invention comprise detecting all or part, including epitopes, of macromolecules associated with a virus. The macromolecules of the invention include proteins, glycoproteins (e.g., envelope glycoproteins), peptides, and polypeptides associated with said virus. The invention also provides for methods of diagnosing a subject infected with a virus utilizing MIPs, in addition to diagnostic kits.

### CROSS REFERENCE TO RELATED APPLICATIONS

The present application claims priority to U.S. Provisional Application Nos. 61/169,450, filed on Apr. 15, 2009, and 61/222,165, filed on Jul. 1, 2009, the disclosures of both of which are specifically incorporated herein by reference in their entirety.

### FIELD OF THE INVENTION

The invention described herein generally relates to molecularly imprinted polymers (MIPs) that are capable of binding to human immunodeficiency virus (HIV), including methods and kits for detecting, identifying and/or quantifying HIV utilizing MIPs.

### BACKGROUND OF THE INVENTION

The human immunodeficiency virus (HIV) is the cause of acquired immune deficiency syndrome (AIDS) (Barre-Sinoussi, F., et al., 1983, Science 220:868-870; Gallo, R., et al., 1984, Science 224:500-503). HIV transmission readily occurs through exposure of the oral, rectal, or vaginal mucosa to the virus during sex, by inoculation with contaminated blood products, through the use of contaminated equipment during injection drug use, by maternal circulation, or by breast feeding. Spread of the disease is facilitated by the long latency period of the virus. Since individuals with HIV infections can be asymptomatic for many years before they develop severe immunodeficiency (AIDS), these infected, asymptomatic individuals can unknowingly transmit the virus.

Complications of HIV-1 infection and acquired immunodeficiency syndrome are a primary cause of morbidity and mortality associated with HIV-1 infection, and significantly reduce the quality of life for people living with AIDS. Although the exact mechanism by which the virus causes complications is not completely understood, it is known that some complications are related to the immune deficiency, whereas others are a secondary consequence of infection with the virus itself.

Early diagnosis of HIV-1 complications would permit a patient and doctor to make decisions about management of their disease that may ultimately improve their quality of life and their ability to live with the disease Immunoassays for the routine detection of HIV-1 infection are known in the art. HIV-1 disease progression can be routinely monitored by measuring the number of circulating CD4 lymphocytes and the quantity of viral RNA in the blood. Alternatively, progression can be monitored by measuring the HIV-1 Gag protein p24 in biologic samples, though this may be less accurate gauge of viral activity than measurements of viral RNA. However, to date there exists a need for low-cost, easy-to-use tests wherein HIV-1 can be routinely detected, diagnosed, and monitored. The invention disclosed herein addresses these and other needs by developing a robust MIPs-based test for HIV-1.

MIPs are engineered cross-linked polymers that exhibit high affinity and selectivity towards a single compound or a family of related compounds. MIPs are able to bind analytes even when these are present in chemically complex solutions or matrices (e.g., plasma, urine, muscle tissue, food matrices, environmental samples, process solutions etc). An important strength of MIPs is that they are able to bind to trace levels of a target molecule, in the presence of large excesses of other compounds that have similar physico-chemical properties. MIPs have a selective synthetic recognition site (or imprint), which is sterically and chemically complementary to a particular target or class of targets. MIPs are economical and fast to produce and are robust and stable under storage. They can be used at elevated temperatures, in organic solvents and at extreme pH values. They also display a higher sample load capacity than is typical for immunoaffinity based sorbents. This results in higher recoveries for analytical applications and suitability of using these sorbents for semi-preparative or preparative scale separations.

Molecular imprinting involves arranging polymerizable functional monomers around a template (for example, a pseudo-target molecule, an analog of the target molecule, all or portion of the actual target molecule, etc., followed by polymerization and template removal. The MIP-template arrangement is typically achieved by: (i) non-covalent interactions (e.g., H-bonds, ion pair interactions) or (ii) reversible covalent interactions. After template removal, these molecularly imprinted polymers can recognize and bind to the actual target molecule.

MIPs hold several advantages over antibodies for diagnostics and sample analysis, due to their controlled synthesis and remarkable stability. Molecular imprinting originates from the concept of creating tailor-made recognition sites in polymers by template polymerization (Mosbach K. et al., Bio/Technology, 1996, 14, 163-170; Ansell R. J. et al., Curr. Opin. Biotechnol., 1996, 7, 89-94; Wulff G. Angew. Chem. Int. Ed. Engl., 1995, 34, 1812-32; Vidyasankar S. et al., Curr. Opin. Biotechnol., 1995, 6, 218-224; and Shea K. J, Trends In Polymer Science, 1994, 2, 166-173). Molecularly imprinted polymers have demonstrated remarkable recognition properties that were successfully applied in various fields such as drug separation (Fischer L., et al., J. Am. Chem. Soc, 1991, 113, 9358-9360; Kempe M, et al., J. Chromatogr., 1994, 664, 276-279; Nilsson K., et al., J. Chromatogr., 1994, 680, 57-61), receptor mimics (Ramstrom O., et al., Tetrahedron: Asymmetry, 1994, 5, 649-656; Ramstrom O., et al., J. MoI. Recogn., 1996, 9, 691-696; Andersson L. L, et al., Proc. Natl. Acad. Sci., 1995, 92, 4788-4792; Andersson L. L, Anal. Chem., 1996, 68, 111-117), bio-mimetic sensors (Kriz D., et al., Anal. Chem., 1995, 67, 2142-2144), antibody mimics (Vlatakis G., et al., Nature, 1993, 361, 645-647), template-assisted synthesis (Bystrom S. E., et al, J. Am. Chem. Soc, 1993, 115, 2081-2083), and catalysis (Muller R., et al., Makromol. Chem., 1993, 14, 637-641; Beach J. V., et al., J. Am. Chem. Soc, 1994, Vol. 116, 379-380).

The great potential embodied in MIPs resulted in numerous inventions for analytical devices and methods of detection of various targets, based on molecular imprinting, reviewed by Ye and Haupt (Anal. Bioanal. Chem. 2004, 378,1887-1897). Some examples of MIP-based sensors are described in U.S. Pat. Nos. 5,587,273, 6,680,210, 6,833,274, 6,967,103 and 6,461,873. Using MIPs combined with displacement of analyte-marker conjugate was shown to be practical in several laboratories (Vlatakis G. et al., Nature, 1993, 361, 645-647, Levi et al., 1997, Anal. Chem. 69. 2017-2021; Nathaniel T. et al., J. Am. Chem. Soc. 2005, 127, 5695-5700; Nicholls C. et al, Biosens. Bioelec, 2006, 21, 1171-1177).

To date, molecular imprints have had limited application to the binding of larger molecules including macromolecules. Synthetic polymers which selectively bind amino acid derivatives and peptides were created using the target amino acid derivative or peptide as a template (Kemp, 1996, Anal. Chem. 68:1948-1953). Imprints have also been created which bind to nucleotide derivatives (Spivak and Shea, 1998, Macromolecules 31:2160-2165). Ionic molecular images of polypeptides have been created by mixing a matrix material with the intact polypeptide chain to be bound by the molecular image (U.S. Pat. No. 5,756,717). Molecular imprints of cytochrome c, hemoglobin and myoglobin, respectively, have been prepared by polymerizing acrylamide in the presence of each intact protein. An imprint of horse myoglobin selectively bound horse myoglobin from a mixture of proteins including whale myoglobin (U.S. Pat. No. 5,814,223).

Although the methods of molecular imprinting have shown limited success at selectively binding macromolecules, the methods have not been utilized to detect or identify viruses. These shortcomings in the art are overcome by the invention described below, which in one aspect provides MIPs useful for detecting, identifying and/or quantifying the virus in a sample or on a target area. Generally, the imprint compositions of the invention described below comprise a matrix material defining an imprint of a template virus. Potential advantages of MIP-based materials include: specificity comparable to an antibody or a biorecognition element; robustness and stability under extreme chemical and/or physical conditions; and an ability to design recognition sites for target molecules that lack suitable biorecognition elements.

### SUMMARY OF THE INVENTION

One embodiment of the invention described herein provides molecularly imprinted polymers (MIPs) capable of specifically binding to a virus.

Another embodiment of the invention provides methods of detecting and/or identifying a virus, comprising contacting said virus with one or more MIPs.

Yet another embodiment of the invention provides methods for diagnosing a subject infected with a virus, comprising contacting a biological sample obtained from said subject with one or more MIPs, and detecting and/or identifying the presence of said virus in said biological sample.

In one embodiment of the invention, the virus can be selected from the group consisting of HIV-1, HIV-2, HTLV-1, HTLV-2, HBV, HPV, HCV, and SIV.

In one embodiment, the MIPs are capable of binding to all or a portion of a macromolecule unique to said virus.

In another embodiment of the invention, the MIPs are capable of binding to an epitope of said viral macromolecule.

In one embodiment of the invention, the macromolecules can be selected from the group consisting of proteins, glycoproteins (e.g., envelope glycoproteins), peptides and polypeptides.

In one embodiment of the invention, the MIPs are capable of binding to all or a portion of HIV-1.

In one embodiment of the invention, the macromolecule associated with HIV-1 is the envelope glycoprotein.

In one embodiment of the invention, the macromolecule is the outer domain (OD) of HIV-1 envelope glycoprotein.

In another embodiment of the invention, the MIPs are capable of binding to amino acid sequence comprising SEQ ID NO:1 (CSGKLIC), and/or portions thereof.

In one embodiment of the invention, the MIPs are coupled with transduction elements such that a measurable signal is produced in response to binding of MIPs to said virus.

In one embodiment of the invention, the MIPs are coupled with transduction elements such that a measurable signal is produced in response to binding of MIPs to all or a portion of said macromolecule.

In one embodiment of the invention, the signal associated with the transduction element is selected from the group consisting of colorimetric, fluorescence, radioactive and enzymatic.

In one embodiment of the invention, the biological sample is selected from the group consisting of biological fluids, tissue extracts and tissues.

In one embodiment of the invention, the biological fluid can be selected from the group consisting of blood, cerebrospinal fluid, serum, plasma, urine, nipple aspirate, fine needle aspirate, tissue lavage, saliva, sputum, ascites fluid, semen, lymph, vaginal pool, synovial fluid, spinal fluid, amniotic fluid, breast milk, pulmonary sputum or surfactant, urine, fecal matter, fluids collected from any of liver, kidney, breast, bone, bone marrow, testes, brain, ovary, skin, lung, prostate, thyroid, pancreas, cervix, stomach, intestine, colorectal, bladder, colon, nares and uterine, head and neck, nasopharynx tumors, and other liquid samples of biologic origin.

In one embodiment of the invention, the tissues can be selected from the group consisting of liver, kidney, breast, testes, brain, ovary, skin, head and neck, lung, prostate, thyroid, pancreas, cervix, stomach, intestine, colorectal, bladder, colon, uterine, and other tissue of interest.

In one embodiment of the invention, the tissue extracts can be selected from the group consisting of liver, kidney, breast, testes, brain, ovary, skin, head and neck, lung, prostate, thyroid, pancreas, cervix, stomach, intestine, colorectal, bladder, colon, uterine extracts and other tissue extracts of interest.

One embodiment of the invention provides for a method of diagnosing a patient infected with HIV-1, comprising contacting a biological sample obtained from said patient with one or more MIPs, and detecting and/or identifying the presence of a macromolecule unique to HIV-1 in said biological sample.

One embodiment of the invention provides for a method of detecting or identifying HIV-1 utilizing MIPs that are capable of binding to all or a portion of a macromolecule associated with HIV-1.

Another embodiment of the invention provides for a method of detecting or identifying HIV-1 utilizing MIPs that are capable of binding to all or portion of the envelope glycoprotein.

Another embodiment of the invention provides for a method of detecting or identifying HIV-1 utilizing MIPs that are capable of binding to all or a portion of the outer domain of HIV-1 envelope glycoprotein.

Another embodiment of the invention provides for a method of detecting or identifying HIV-1 utilizing MIPs that are capable of binding to amino acid sequence comprising SEQ ID NO:1 (CSGKLIC), and/or portions thereof.

In one embodiment of the invention, a detectable signal is produced upon binding of all or a portion of a macromolecule associated with HIV-1.

In one embodiment of the invention, a detectable signal is produced upon binding of HIV-1 with MIPs that are capable of binding to all or a portion of the envelope glycoprotein.

In one embodiment of the invention, a detectable signal is produced upon binding of HIV-1 with MIPs that are capable of binding to all or a portion of the outer domain of HIV-1 envelope glycoprotein.

In another embodiment of the invention, a detectable signal is produced upon the binding of HIV-1 with MIPs that are capable of binding to amino acid sequence comprising SEQ ID NO:1 (CSGKLIC), and/or portions thereof.

One embodiment of the invention provides for methods of diagnosing a patient with HIV-1 infection.

In one embodiment of the invention, the method of diagnosing a patient with HIV-1 infection comprises contacting a biological sample obtained from said patient with one or more MIPs that are capable of binding to a macromolecule associated with HIV-1.

In another embodiment of the invention, the method of diagnosing a patient with HIV-1 infection comprises measuring the detectable signal produced upon binding of HIV-1 in the biological sample obtained from said patient to MIPs that are capable of binding to a macromolecule associated with HIV-1.

In one embodiment of the invention, the method of diagnosing a patient with HIV-1 infection comprises contacting a biological sample obtained from said patient with one or more MIPs that are capable of binding to all or portion of the envelope glycoprotein.

In one embodiment of the invention, the method of diagnosing a patient with HIV-1 infection comprises contacting a biological sample obtained from said patient with one or more MIPs that are capable of binding to all or portion of the outer domain of HIV-1 envelope glycoprotein.

In another embodiment of the invention, the method of diagnosing a patient with HIV-1 infection comprises measuring the detectable signal produced upon binding of HIV-1 in the biological sample obtained from said patient to MIPs that are capable of binding to all or portion of the envelope glycoprotein.

In another embodiment of the invention, the method of diagnosing a patient with HIV-1 infection comprises measuring the detectable signal produced upon binding of HIV-1 in the biological sample obtained from said patient to MIPs are capable of binding to amino acid sequence comprising SEQ ID NO:1 (CSGKLIC), and/or portions thereof.

In one embodiment of the invention, the method of diagnosing a patient with HIV-1 infection comprises contacting a biological sample obtained from said patient with one or more MIPs that are capable of binding to amino acid sequence comprising SEQ ID NO:1 (CSGKLIC) and/or portions thereof.

In another embodiment of the invention, the method of diagnosing a patient with HIV-1 infection comprises measuring the detectable signal produced upon binding of HIV-1 in the biological sample obtained from said patient to MIPs that are capable of binding to amino acid sequence comprising SEQ ID NO:1 (CSGKLIC), and/or portions thereof.

One embodiment of the invention provides methods for determining the onset, progression, or regression of an infection associated with HIV-1 in a subject, wherein a biological sample obtained from a subject is screened for said virus by contacting said biological sample with one or more MIPs.

One embodiment of the invention provides a kit comprising one or more MIPs for detecting or identifying HIV-1.

Another embodiment of the invention provides methods to detect the presence of HIV-1 on a target area, comprising contacting the target area with one or more MIPs.

In one embodiment, the target area includes environmental surfaces, such as in hospitals, sports equipment and medical devices.

In another embodiment of the invention, the target area can be selected from the group consisting of bed railing, door knobs and computer keyboards.

### BRIEF DESCRIPTION OF THE DRAWINGS

FIG. 1 illustrates a method of generating MIPs.

FIG. 2A illustrates a schematic representation of generating MIPs to a macromolecule associated with a virus.

FIG. 2B illustrates a schematic representation of detecting a virus utilizing MIPs of the present invention.

### DETAILED DESCRIPTION OF THE INVENTION

The invention described herein provides molecularly imprinted polymers (MIPs) capable of binding to the human immunodeficiency virus (HIV), and methods for detecting HIV utilizing such MIPs. Additionally, the invention provides methods and kits for detecting, identifying and/or quantifying HIV in biological samples, including, but not limited to, tissues, tissue extracts, and biological fluids. MIPs of the invention may be used to identify and/or quantify HIV on target surfaces and in liquid samples. MIPs of the invention may be used anywhere detection of HIV is desired, for example in home, clinic, doctor's office, hospital bedside, factory, or field. The methods can be used for detecting HIV in a variety of biological, environmental and industrial samples without the need for complicated sample preparation procedures, and thus are also suitable for use by untrained personnel even in field conditions. Further, the invention provides methods for diagnosing an infection associated with a virus utilizing MIPs.

The MIPs of the invention can be prepared in accordance with any technique known to those skilled in the art using a virus particle or a portion thereof as a template molecule. These methods include covalent imprinting (Wulff, 1982, Pure & Appl. Chem., 54, 2093-2102) whereby the monomers are covalently attached to the template and polymerized using a cross-linker. Subsequently, the template is cleaved from the polymer leaving template-specific binding cavities. Alternatively, a non-covalent imprinting method such as disclosed by U.S. Pat. No. 5,110,833, the portions of which disclose non-covalent imprinting are specifically incorporated herein by reference, may be used. In non-covalent imprinting methods, the monomers interact with the target molecule by non-covalent forces, and are then polymerized via a cross-linker to form target specific binding sites after removal of the target molecule. Combinations and variations on these methods may be used to construct thin molecularly imprinted films and membranes (Hong et al., 1998 Chem. Mater., 10, 1029-1033); imprinting on the surface of solid supports (Blanco-López, et. al., 2004, Anal. Bioanal. Chem., 378, 1922-1928; Sulitzky C. et al., 2002 Macromolecules, 35, 79-91); and microspheres (Ye et al., 2000, Macromolecules, 33, 8239-8245). Further, methods for preparing MIPs are described in U.S. Pat. Nos. 4,406,792, 4,415,655, 4,532,232, 4,935,365, 4,960,762, 5,015,576, 5,208,155, 5,310,648, 5,321,102, 5,372,719, 5,786,428, 6,063,637, and 6,593,142, the portions of all of which disclosing the methods for preparing MIPs are specifically incorporated herein by reference.

One example of molecular imprinting involves making a polymer cast of a template molecule, wherein the template includes, but is not limited to, an epitope. The process of making the polymer cast involves dissolving the template molecule to be imprinted in a suitable solvent. Normally, an imprint composition comprising a co-monomer, cross-linking monomer and a polymerization initiator is added to the solvent comprising the desired template to form a reaction mixture. Radiation (photochemical or ionizing) or thermal energy is then applied to the reaction mixture, to initiate the polymerization process, ultimately resulting in the formation of a solid polymer. The resulting polymer may be processed using conventional polymer processing technologies, assuming those processes do not alter the structure of the molecularly imprinted sites. The imprinted molecule is extracted using methods appropriate for dissociating the template molecule from the polymer. Details of template molecule dissociation from the polymer are dependent upon the nature of the chemical interaction between the target molecule and the polymer binding site. The polymer dissociated from the template molecule possesses binding sites optimized for the structural and electronic properties of such template molecule.

Preferably, the conditions under which the template molecule is imprinted are similar or identical to the conditions under which the macromolecule is to be captured by the MIPs of the invention. For instance, if the macromolecule is to be captured under denaturing conditions, then the template molecule should be imprinted under the same denaturing conditions. Similarly, if the macromolecule is to be captured under native conditions, then the template molecule should be imprinted under the same native conditions. Native and denaturing conditions are well-known to those of skill in the art. Many heat-sensitive compounds that can be used to make imprint compositions according to the invention are known in the art and include, by way of example and not limitation, hydrogels such as agarose, gelatins, moldable plastics, etc. Examples of other suitable hydrogels are described in U.S. Pat. No. 6,018,033, U.S. Pat. No. 5,277,915, U.S. Pat. No. 4,024,073, and U.S. Pat. No. 4,452,892, the portions of all of which that relate to imprinting are incorporated herein by reference.

Suitable non-limiting examples of monomers that can be used for preparing a polymer of the present invention include methylmethacrylate, other alkyl methacrylates, alkylacrylates, ally or aryl acrylates and methacrylates, cyanoacrylate, styrene, α-methyl styrene, vinyl esters, including vinyl acetate, vinyl chloride, methyl vinyl ketone, vinylidene chloride, acrylamide, methacrylamide, acrylonitrile, methacrylonitrile, ethylene glycol diacrylate, pentaerythritol dimethacrylate, pentaerythritol diacrylate, N,N′-methylenebisacrylamide, N,N′-ethylenebisacrylamide and N,N′-(1,2-dihydroxyethylene)bisacrylamide. Depending upon the choice of the monomers used, the polymer particles will have a variety of physical and mechanical properties, such as hydrophobicity/hydrophilicity, mechanical strength and ease or resistance to swelling in the presence of solvents.

The MIPs of the invention may take a variety of different forms. For example, they may be in the form of individual beads, disks, ellipses, or other regular or irregular shapes (collectively referred to as “beads”), or in the form of sheets. Each bead or sheet may comprise imprint cavities of a single template molecule, or they may comprise imprint cavities of two or more same or different template molecules. In one embodiment, the MIPs comprise imprint cavities of a plurality of different template molecules arranged in an array or other pattern such that the relative positions of the imprint cavities within the array or pattern correlate with their identities, i.e. the identities of the template molecules used to create them. Each position or address within the array may comprise an imprint cavity of a single template molecule, or imprint cavities of a plurality of different template molecules, depending upon the application. Moreover, the entire array or pattern may comprise unique imprint cavities, or may include redundancies, depending upon the application.

In one embodiment, the invention provides methods of manufacturing matrix materials comprising the imprint compositions. Such matrix materials include, but are not limited to, substances that are capable of undergoing a physical change from a fluid state to a semi-solid or solid state. In the fluid state, the particles of a matrix material move easily among themselves, and the material retains little or no definite form. A matrix material in the fluid state can be mixed with other compounds, including template molecules. In the semi-solid or solid state, the matrix materials are capable of forming and retaining cavities that complement the shape of template molecules. Examples of such matrix materials include heat sensitive hydrogels such as agarose, polymers such as acrylamide, and cross-linked polymers.

In one embodiment of the invention, the MIPs are capable of binding to virus by way of binding to a macromolecule associated with the virus. Generally, MIPs can specifically bind to all or a portion of the macromolecules including, but not limited to, epitopes associated with the virus.

The template molecule of the invention described herein can be selected from the group consisting of all or a portion of the virus, a macromolecule or a portion thereof associated with the virus, and analogs thereof. The portion to which the template molecule corresponds to may be an internal portion of the macromolecule and/or an external portion, and/or a terminal portion of the macromolecule. Alternatively, the portion may be a side-group or modification of the macromolecule, such as a polysaccharide group of a glycoprotein macromolecule, or a portion thereof.

An embodiment of the invention includes identifying potential conserved regions of one or more HIV proteins, for example gag, pol, reverse trascriptase (RT), the gp120 and gp41 glycoproteins and determining the best epitopes for the development of the MIPs. The methods to manufacture MIPs include 1) using epitope template imprinting (Buechel and Sellergren 1999; Titirici et al. 2004; Nishino et al. 2006) directed at the conserved regions of HIV-1 proteins, for example the gp120 and gp41 glycoproteins, that makes use of short peptide sequences as templates to generate binding sites representative of the larger peptides or proteins. Access of the target to the sites is obtained by immobilizing it on a “void creator” such as porous silica gel, or colloidal silica which post-polymerization, is removed together with the peptide template in an etching step. Alternatively, the epitope may be conjugated to a polymeric protein surrogate that is then removed for liberation of the epitope-selective sites; and 2) hierarchical imprinting approach permitting the identification of the epitope-imprinted sites to HIV-1 proteins, for example the gp120 and gp41 glycoproteins, accessible surfaces (Titirici et al. 2003) that allows porous materials with different morphologies to be prepared. Commonly, an inorganic material serves as template for the synthesis of organic materials of defined morphology. Porous silica has been used as a sacrificial template for the synthesis of mesoporous organic polymer networks (Titirici et al. 2002). This occurs by filling the pore system of porous silica particles with organic monomers and initiator followed by heating to induce polymerization. This produces an inorganic/organic composite material which is finally freed from silica by etching to yield a polymeric replica of the original pore system of the silica template.

Binding of an HIV protein to the MIP of the invention can be detected by using any suitable transduction elements known in the art, for example, colorimetric transduction. In embodiments of this type, the MIPs may have a dye-labeled peptide selectively bound in the imprinted receptor sites. When a solution of the target analyte (unlabeled amino acid sequence) is present, the dye-labeled peptide will be displaced from the MIP receptor sites into solution. This will cause an obvious and unambiguous color change indicating to the user that the target analyte is present and allowing for a quick determination of the presence of HIV-1 or any other virus in a matter of seconds without need for special training or equipment. One method might entail obtaining saliva, urine, tears, sweat, etc. from a subject and applying the MIPs solution and detecting a color change. This can be done easily in the field by dispensing the MIPs in user-friendly sterile single-use containers similar to what is used to administer eye-drops.

Viruses that can be captured, detected, identified and/or quantified using the MIPs of the invention described herein include, but are not limited to, the Human Immunodeficiency Virus Type 1 (HIV-1), Human Immunodeficiency Virus Type 2 (HIV-2), Human T-lymphotropic Virus Type 1 (HTLV-1), Human T-lymphotropic Virus Type 2 (HTLV-2), Hepatitis B Virus (HBV), Human Papilloma Virus (HPV), Human Herpes Virus (HHV), Hepatitis C Virus (HCV) and Simian Immunodeficiency Virus (SIV). In one embodiment, MIPs and methods of the invention may be used to detect Human Immunodeficiency Virus-1 (HIV-1).

Typically, viruses will be captured, identified and quantified by the interaction of a MIP of the invention and a macromolecule associated with the virus. Macromolecules associated with viruses that can be detected, identified and/or quantified using the MIPs of the invention include any type of macromolecule from which a template molecule can be designed and constructed according to the principles taught herein. Virtually any type of macromolecule can be detected, identified and/or quantified using the methods and compositions of the invention. Non-limiting examples include polysaccharides, proteins, glycoproteins, peptidoglycans, lipoproteins, peptides, polypeptides, and polynucleotides, and other macromolecular targets that will be apparent to those of skill in the art.

The target molecules can be selected from, but are not limited to, proteins e.g. envelope glycoprotein and capsid. For example, the envelope gene product of HIV is synthesized as a gp160 precursor molecule, which is subsequently processed into the external envelope protein gp120 and the transmembrane protein gp41. The HIV-1 envelope gp120 glycoprotein has shown remarkable diversity, glycosylations, and conformational flexibility, which allows it to evade antibody-mediated neutralization. Despite this complexity, the HIV-1 Env must contain conserved determinants that mediate CD4 binding. The core of gp120 glycoprotein from HIV-1 is comprised of three major structural domains: the outer domain, the inner domain, and the bridging sheet. The outer domain is exposed on the HIV-1 envelope glycoprotein trimer and contains binding surfaces for neutralizing antibodies such as 2G-12, immunoglobulin G1b12, and anti-V3 antibodies (Yang et al., J. Virol. 2004. 78:12975-12986; Zhou et al., J. Virol. 2009. 83 (10):5077-86). Residues 650-661 of gp120 glycoprotein encompass a charged helix; Residues 662-667, referred to as the conserved epitope ELDKWA, is the epitope recognized by antibodies that neutralize HIV-1 entry in epithelial and CD4⁺-mononucleated cells; and Residues 668-685, are hydrophobic Trp-rich sequence that stabilizes the structure of the galactose binding site. Studies show that the best-exposed epitopes that occur on the surface of intact virions are found in the V3, CD4bd, and C5 regions of gp120 and in antigenic cluster I of gp41 (spanning amino acids 579 to 613 of the envelope) (Moore et al., J. Virol. 1994. 68 (1):469-84; Nyambi et al., J. Virol. 2000. 74 (15):7096-107; Nyambi et al., J. Immunol. Methods. 2001. 253 (1-2):253-262; Nyambi et al., J. Virol. 2008. 72 (11): 9384-91; Nyambi et al., J. Immunol. Methods. 2001. 253 (1-2):253-262). Additionally, the HIV-1 gp41 disulfide-bonded loop/chain reversal region is a critical gp120 contact site; therefore, it is also likely to play a central role in fusion activation by linking CD4 plus chemokine receptor-induced conformational changes in gp120 to gp41 fusogenicity (Maerz et al., J. Virol. 2001. 75 (14):6635-44).

The above-mentioned conserved regions of gp120 and gp41 have been well characterized with the goal of developing vaccines against HIV-1. To date, no effective vaccine has been developed. However, such conserved regions are good targets for MIPs of the invention described herein. For example, antibody b12 IgG1 has been found to bind to a conformationally invariant surface that overlaps a distinct subset of the CD4-binding site. The crystal structure for the b12 antibody protein has been well characterized. Therefore, MIPs that mimic the b12 epitope can be designed that will bind to the conserved surface and, upon binding, change color signaling the presence of HIV-1. Another example is the development of human monoclonal antibody 41-7 IgG1(k) directed against gp41. This antibody has been mapped to a conserved seven-amino acid sequence, CSGKLIC (SEQ ID NO:1) located within the N-terminal part of gp41. Further, a 231-residue fragment of the HIV-1 R3A TA1 gp120 envelope (residues 252-482) has been shown to bind b12 (Wu et al., J Virol. 2009. 83 (10):5077-86, Laakso et al., PLoS Pathog. 2007. 24; 3 (8): e117; Nolan et al., J Virol. 2008 82 (2):664-73).

Additionally, epitopes within gp120 and gp41 that have been shown to be highly conserved within various HIV-1 strains can be utilized to produce MIPs. For example, the HIV-1 envelope gp41 residues 650-685, involved in the initial step of interaction between the virus and epithelial cells, are highly conserved among HIV-1 isolates (Alsen and Bomsel, J. Biol. Chem. 2002. 277 (28):25649-59). The third variable region (V3) of gp120 has been shown to be at least partially exposed during various stages of the infectious process, is immunogenic in essentially all HIV+ subjects, and is capable of inducing Abs able to neutralize a broad array of primary isolates (Zolla-Pazner, S. Hum Antibodies. 2005; 14 (3-4):69-72). The D19 epitope contained within the third variable (V3) domain of gp120 was found to be conserved in the majority (23/29; 79.3%) of the subtype-B strains tested, as well as in selected strains from other genetic subtypes (Lusso et al., J. Virol. 2005. 79 (11):6957-68). Segments of V1, V2, and V3 loops of HIV-1 gp120 glycoprotein have been found to be exposed (Moore et al., J. Virol. 1994. 68 (1):469-84; Pinter et al., J. Viol. 1993. 67(9):5692-7). Such exposed regions can serve as good targets for MIPs of the invention described herein.

The gp120 association site includes conserved aromatic and hydrophobic residues, Leu-593, Trp-596, Gly-597, and Trp-610, on either side of the disulfide-bonded loop and a basic residue, Lys-601, within the loop. This region was found to link CD4 and chemokine receptor-induced conformational changes in gp120 to gp41 fusion activation (Maerz et al., J. Virol. 2001. 75 (14):635-44). Epitopes in the first conserved region (109-113) and the third conserved/fourth variable regions (376-380, 382-384, 395-400) are shown to be more “surface-exposed” (Fouts et al., 1999. Vaccine 13 (6):561-9). Peptide sequences engineered to comprise the above-mentioned conserved residues to structurally resemble native confirmation of such region of envelope glycoprotein can be utilized as templates in the manufacturing MIPs of the present invention. It has also been shown that Env proteins containing mutations within gp41 that expose multiple discontinuous neutralization epitopes on diverse HIV-1 Env proteins could act as a scaffold for presentation of such conserved domains, and may aid in developing methods to target antibodies to such regions (Blish et al., PLoS Med. 2008 January; 5 (1): e9).

Examples of epitopes within the envelope glycoprotein of HIV-1 include, but are not limited to, GPGRAFY (SEQ ID NO:2), RILAVERYLKD (SEQ ID NO:3), ELDKWA (SEQ ID NO:4), ELDKWAG (SEQ ID NO:5), GPCGRAFY (SEQ ID NO:6), ELLDKWAG (SEQ ID NO:7), RIVALVERYLKD (SEQ ID NO:8), GPGRAFY (SEQ ID NO:9) (Lu et al., Allergy and Immunolgy, 2000. 121:80-84), ELDKWAS (SEQ ID NO:10) (Ho et. al., Vaccine, 2005. 23 (13):1559-73) RKSIRIQRGPGRAFVTIG (SEQ ID NO:11), QRGPGR (SEQ ID NO:12) (Tugarinov et al., Structure. 2000. 8 (4):385-95) CVAMKCSSTE (SEQ ID NO:13), SSTGNNTTSK (SEQ ID NO:14), STSTTTTTPT (SEQ ID NO:15), DQEQEISEDT (SEQ ID NO:16), PCARADNCSG (SEQ ID NO:17), LGEEETINCQ (SEQ ID NO:18), FNMTGLERDK (SEQ ID NO:19), KKQYNETWYS KDVVC (SEQ ID NO:20), ETNNS (SEQ ID NO:21), TNQTQCYMNH (SEQ ID NO:22), CKRPGNKIVK (SEQ ID NO:23), QIMLMSGHVF (SEQ ID NO:24), HSHYQPINKR (SEQ ID NO:25), PRQAWC (SEQ ID NO:26), DDYQEITLNVTE (SEQ ID NO:27), SEDTPCARA (SEQ ID NO:28), GEETINCQ (SEQ ID NO:29), FNMTGL (SEQ ID NO:30), YSKDVVCET (SEQ ID NO:31), DTNYSGFAPNCS (SEQ ID NO:32), LMSGHVFHSHYQ (SEQ ID NO:33), SGHVFHSHYQ (SEQ ID NO:34), DDYQE1TLNVTE (SEQ ID NO:35), DDYQEPTLNVTE (SEQ ID NO:36), GDYSELALNVTE (SEQ ID NO:37), NDTDPCIKT (SEQ ID NO:38), NDTDPCIQL (SEQ ID NO:39), NETSSCIAQ (SEQ ID NO:40), GEEETINCQ (SEQ ID NO:41), GEEEDINCE (SEQ ID NO:42), REEDTDNCQ (SEQ ID NO:43), EQEQMIGCK (SEQ ID NO:44), FNMTGL (SEQ ID NO:45), ENDTGL (SEQ ID NO:46), YSKDVVCET (SEQ ID NO:47), YSADVVCET (SEQ ID NO:48), YSKDVVCES (SEQ ID NO:49), YSTDLVVCET (SEQ ID NO:50), DTNYSGFEPNCS (SEQ ID NO:51), DTNYSGFMPKCS (SEQ ID NO:52) (McKnight et al., J. Virol. 1996. 70 (7):4598-4606), INCTRPNNNTRKSIR (SEQ ID NO:53), PNNNTRKSIRIQRGP (SEQ ID NO:54), RKSIRIQRGPGRAFV (SEQ ID NO:55), RKSIRIQRPGRAFV (SEQ ID NO:56), IQRGPGRAFVTIGKI (SEQ ID NO:57), GNMTQAHCNISRAKW (SEQ ID NO:58), AHNCISRAKWNNTLK (SEQ ID NO:59), GRAFVTIGKIGNMRQ (SEQ ID NO:60), TIGKIGNMRQAHCNI (SEQ ID NO:61), VKIEPLGVAPTKAKR (SEQ ID NO:62), LGVAPTKAKRRVVQR (SEQ ID NO:63), and EVGKAMYAPPISHQI (SEQ ID NO:64), (Ohlin et al., Clin Exp Immunol. 1992 August; 89 (2):290-5, (Bahraoui et al., AIDS Res Hum Retroviruses. 1989 August; 5 (4):451-63).

Accordingly, for diagnostic purposes a MIP can be designed to one or more of the amino acid sequences listed above, including all or portion of the HIV-1 R3A TA1 gp120 envelope (residues 252-482) that has been shown to bind b12) and upon binding change color, thereby signaling the presence of HIV-1. Beyond HIV, there are many proteins for other viruses that have been characterized which proteins or fragments thereof can be useful targets for MIPs-based diagnostics. Non-limiting examples of such proteins include the major structural protein (sHBsAg) of HBV envelope, the capsid proteins L1 and L2 of HPV, the core and envelope proteins of HCV, and the SIV surface glycoprotein gp120.

In general, the structural units of the macromolecule to which the template molecule corresponds are contiguous within the primary structure of the macromolecule. If one of skill in the art can identify a terminus or termini in the primary structure of the macromolecule, then a preferred template molecule corresponds to a template that includes a terminus of the macromolecule. Alternatively, the portion of the macromolecule to which the template molecule corresponds can be expressed in size as a fraction of the size of the entire macromolecule. For example, template molecules can correspond to a portion of the macromolecule that consists of from 1% to 5%, from 1 to 10%, from 1 to 15%, from 1 to 20%, from 1 to 25%, from 1 to 30%, from 1 to 35%, from 1 to 40%, from 1 to 50%, from 1 to 60%, from 1 to 70%, from 1 to 80%, from 1 to 90%, from 1 to 95%, or from 1 to 99% of the structure of the entire macromolecule. Preferably, template molecules have a primary structure that corresponds to a contiguous portion of the primary structure of the macromolecule.

If the macromolecule is a polypeptide, the template molecule can correspond to a portion of the polypeptide that consists of a sequence of amino acids selected from the primary sequence of the polypeptide or an analog thereof. For instance, the portion of the polypeptide can consist of a range of amino acids from the primary structure of the polypeptide consisting of from 1 to 50 amino acids, from 2 to 40 amino acids, from 3 to 30 amino acids, from 3 to 15 amino acids, from 3 to 10 amino acids, from 4 to 10 amino acids, from 4 to 9 amino acids, from 4 to 8 amino acids, from 4 to 7 amino acids, or from 5 to 7 amino acids. Preferred portions of the macromolecule are those that consist of a contiguous sequence of amino acids from the primary structure of the polypeptide.

When the macromolecule is a polynucleotide, the template molecule can be an oligonucleotide having a sequence of nucleotides selected from the primary sequence of the polynucleotide or an analog thereof. If the polynucleotide has n nucleotides, then the selected sequence of nucleotides can have a length from 1 to (n-1) nucleotides. Alternatively, the selected sequence can contain from 1 to 50 nucleotides, 2 to 40 nucleotides, 3 to 30 nucleotides, 3 to 15 nucleotides, 3 to 10 nucleotides, 4 to 10 nucleotides, 4 to 9 nucleotides, 4 to 8 nucleotides, 4 to 7 nucleotides, or 5 to 7 nucleotides. Preferably, the selected sequence is a contiguous sequence of nucleotides from the primary sequence of the polynucleotide.

It will be understood that as used herein, the expression “macromolecule” is not intended to place specific size limitations upon the molecules that may be identified with the MIPs of the methods described herein. Rather, macromolecules include molecules that comprise a plurality of structural moieties or analogs thereof such that a template molecule corresponding to at least one of the structural moieties can be used to prepare a molecular imprint capable of binding the macromolecule. In one embodiment of the invention, template molecules corresponding to at least two of the structural moieties can be used to prepare a molecular imprint capable of binding the macromolecule. In one embodiment of the invention, template molecules corresponding to at least three of the structural moieties can be used to prepare a molecular imprint capable of binding the macromolecule. In one embodiment of the invention, template molecules corresponding to at least four of the structural moieties can be used to prepare a molecular imprint capable of binding the macromolecule.

By “analog” is meant a molecule that differs from, but is structurally, functionally, and/or chemically related to the reference molecule. The analog may retain the essential properties, functions, or structures of the reference molecule. Most preferably, the analog retains the steric and electrostatic properties of the reference molecule. Generally, differences are limited so that the structure or sequence of the reference molecule and the analog are similar overall. For example, a peptide analog and its reference peptide may differ in amino acid sequence by one or more substitutions, additions, and/or deletions, in any combination. Other examples of analogs include peptides with minor amino acid variations from the peptides exemplified herein. In particular, peptides containing conservative amino acid replacements, i.e., those that take place within a family of amino acids that are related in their side chains, constitute analogs. A substituted or inserted amino acid residue may or may not be one encoded by the genetic code. An analog of a peptide or polypeptide may be naturally occurring such as an allelic variant, or it may be a variant that is not known to occur naturally. Non-naturally occurring analogs of peptides may be made by direct synthesis, by modification, or by mutagenesis techniques.

The identities of the structural moieties that comprise macromolecules will depend upon the nature of the macromolecule, and may include regions of primary, secondary and/or tertiary structure of the macromolecule. For example, for polypeptide macromolecules the structural moieties may be the individual amino acids composing the polypeptide, or alternatively, if the polypeptide has several structural domains, the structural moieties may be the individual structural domains. For example, a polypeptide may be viewed as being composed of individual amino acids or structural domains as described above and/or saccharide or oligosaccharide structural moieties; a polynucleotide macromolecule may be viewed as being composed of individual nucleotide structural moieties. The macromolecules according to the invention may be derived from virtually any source. They may be obtained from natural sources such as biological samples or from synthetic sources.

One embodiment of the invention provides for methods of manufacturing MIPs specific for HIV-1 utilizing all or portion of HIV-1 as a template molecule.

In one embodiment, methods of the invention encompass manufacturing MIPs specific for HIV-1 comprising generating a template corresponding to all or portion of a macromolecule associated with HIV-1 and utilizing such template to manufacture MIPs.

In one embodiment, the invention provides for methods of identifying HIV-1, comprising utilizing MIPs that are capable of binding to all or a portion of envelope glycoprotein, gp160.

In another embodiment, the invention provides for methods of identifying HIV-1, comprising utilizing MIPs that are capable of binding to an amino acid sequence comprising CSGKLIC (SEQ ID NO:1) and/or fragments thereof.

Similarly, various viruses can be identified and/or detected utilizing a variety of macromolecules, as mentioned above, associated with said viruses as templates to generate MIPs. Non-limiting examples of the macromolecules include, but are limited to capsid or envelope proteins.

Alternatively, the MIPs of the invention are coupled with transduction elements such that a detectable signal is produced in response to binding of MIPs to said template or target molecule.

Suitable examples of transduction elements include, but not limited to, HABA [2(4′-hydroxyazo benzene)-benzoic acid], dyes, fluorescers, fluorescent dyes, radiolabels, magnetic particles, metallic particles, colored particles, metal sols, enzyme substrates, enzymes, chemiluminescers, photosensitizers and suspendable particles.

In some embodiments, the detectable signal may be a visible substance, such as a colored latex bead, or it may participate in a reaction by which a colored product is produced. The reaction product may be visible when viewed with the naked eye, or may be apparent, for example, when exposed to a specialized light source, such as ultraviolet light.

The concentration of template or the target molecule may be indicated by the amount of detectable signal associated with the transduction element.

Additionally, target detection by MIPs may be signaled in a variety of ways. In some cases, the detection signal may be visualized (e.g., luminescence or change in color). One such technique for providing a color change response using MIPs is explained in the document entitled “Molecularly Imprinted Polymer Sensor Aerosol” by George M. Murray, Ph.D., which is incorporated by reference herein in its entirety. A technique for using porphyrins with MIPs to cause a change in absorption/emission of electromagnetic radiation is explained in U.S. Pat. No. 6,872,786, the portion of which that describes the technique for using porphyrins with MIPs is incorporated herein by reference. Some techniques for producing luminescence using MIPs upon target detection are explained in U.S. Pat. No. 6,749,811, the portion of which that describes target detection using a MIP is incorporated herein by reference; A. L. Jenkins et al., Anal. Chem., vol. 71:2, pp. 373-378 (1999); and B. R. Arnold et al., Johns Hopkins APL Technical Digest, vol. 20:2, pp. 190-198 (1999). Any of the preceding techniques can be adapted for use with the MIPs of the present invention.

For example, MIPs in accordance with one aspect of the present invention can be prepared by (A) providing the reaction product of a polymerizable porphyrin derivative and a template molecule; (B) copolymerizing the reaction product of step (A) with one ore more monomers and crosslinking agent to form a polymer; and (C) removing the template molecule from the polymer to provide a molecularly imprinted polymer which exhibits selective binding affinity for the template molecule and undergoes a detectable change in absorption and/or emission of electromagnetic radiation when the target molecule binds thereto. The polymerization reaction mixture for preparation of a MIP therefore constitutes the reaction product of step (A), one or more polymerizable monomers, an effective amount of one or more crosslinking agents to impart a sufficiently rigid structure to the polymer end-product, inert solvent, and a free radical or other appropriate initiator. Mixtures of monomers and crosslinking agents can be used in the polymerization method. The amounts of polymerizable porphyrin, monomer and crosslinking agents can vary broadly, depending on the specific nature/reactivities of the polymerizable porphyrin, monomer and crosslinking agent chosen as well as the specific sensor application and environment in which the polymer/sensor will be ultimately employed. The relative amounts of each reactant can be varied to achieve desired concentrations of porphyrin in the polymer support structure. The solvent, temperature and means of polymerization can be varied in order to obtain polymeric materials of optimal physical or chemical features, for example, porosity, stability, and hydrophilicity. The solvent can also be chosen based on its ability to solubilize all the various components of the reaction mixture.

Further, according to another embodiment of the invention described herein, the MIPs may comprise lanthanide-containing polymeric structures that exhibit selective binding characteristics towards a target to be detected by a sensor device or kit of the invention described herein. The polymerization step comprises co-polymerizing a chelated lanthanide-template complex with one or more cross-linking monomers, and optionally, one or more additional matrix monomers to form a polymer structure. Any of a wide range of lanthanide metal salts capable of dissociating in solution to form a lanthanide ion, and combinations of two or more thereof, are suitable for use in the invention described herein. Examples of suitable lanthanide salts include, but are not limited to, halides, nitrates, perchlorates, and the like, of lanthanum (La), cerium (Ce), praseodymium (Pr), neodymium (Nd), promethium (Pm), samarium (Sm), europium (Eu), gadolinium (Gd), terbium (Th), dysprosium (Dy), holmium (Ho), erbium (Er), thulium (Tm), ytterbium (Yb), and lutetium (Lu). MIPs can be used as part of an optical sensor device to selectively capture the target, by associating such molecules with MIP lanthanide binding sites. Such MIPs act as a source of luminescence, which can be analyzed to determine the amount of target in the solution. Any of a wide range of suitable detectors can be used according to the invention described herein. Non-limiting examples of suitable detectors include a spectrophotometer, spectrometer (gas or mass), photomultiplier tube, monochromator equipped with a CCD camera, filters, the naked eye, combinations of two or more thereof, and the like.

The invention described herein additionally presents a method for forming a reliable chemical sensor platform based on site selectively tagged and templated molecularly imprinted polymers (SSTT-MIP). The SSTT-MIP strategy used in the present method provides a way to form a MIP having a templated site specific for an analyte and at which a reporter molecule can also be attached. In this way, analyte detection can be carried out with a higher efficiency in comparison to methodologies without any provision for such positioning. With this invention, measurement characteristics such as signal-to-background and signal-to-noise ratios are expected to be improved.

The invention also provides molecularly imprinted polymer platforms in which templated sites are formed for specific target molecules. In one embodiment, the polymer platforms can be provided wherein the templated sites have at least one reporter molecule bonded to a reactive group at the site. In one embodiment, the polymer platform comprises xerogels or aerogels. Methods to develop sensors for the detection of a wide variety of targets are described by Bright et al., in U.S Publication No 2005/0227258, the portions of which that describe development of templated sites for specific target molecules, is incorporated herein by reference.

For example, the template is chosen such that it forms bonds with the polymer platform in excess of the number of those which will be formed by the bound target. The template can have at least one additional reactive group so as to be able to bind to the polymer matrix at an additional site relative to an intended target. In one embodiment, the number of reactive groups on the template which link it to the polymer platform, is at least one more than the number of reactive groups on the corresponding target. The removal of the template results in cavities within the polymer platform. Exposed at each templated site are reactive groups which are responsible for target recognition. However, as a consequence of the additional reactive group(s) mentioned above, when the template is cleaved from the cavity, the cavity bears one or more reactive groups in excess of the groups needed to bind the target. The extra group(s) is (are) used to bond with reporter molecules. Once the reporter molecule(s) is (are) bound at the templated site, the absorbance/luminescence from the target bound MIP can be measured and a change in UV, visible or IR absorbance/luminescence properties of the reporter (e.g., absorbance spectra, excitation and emission spectra, excited-state luminescence lifetime and/or luminescence polarization) indicates the presence of target at the templated site. The total change in absorbance/luminescence is generally proportional to the concentration of target molecule in the sample.

The MIPs of the invention can be utilized for diagnosing a subject infected with a virus, comprising contacting a biological sample obtained from said subject with one or more MIPs, and detecting and/or identifying the presence of said virus in said biological sample. The methods of diagnosis comprises measuring the level of all or a portion said virus, or all or a portion of macromolecule associated with said virus in a biological sample or biological fluid obtained from said patient.

The MIPs of the invention can also be utilized for determining the onset, progression, or regression of an infection associated with a virus in a subject, wherein a biological sample obtained from a subject is screened for all or a portion said virus, or all or a portion of macromolecule associated with said virus by contacting said biological sample with one or more MIPs.

As used herein, the phrase “biological sample” encompasses a variety of sample types obtained from a subject and useful in the procedure of the invention. Biological samples may include, but are not limited to, solid tissue samples, liquid tissue samples, biological fluids, aspirates, cells and cell fragments. Specific examples of biological samples include, but are not limited to, solid tissue samples obtained by surgical removal, pathology specimens, archived samples, or biopsy specimens, tissue cultures or cells derived therefrom and the progeny thereof, and sections or smears prepared from any of these sources. Non-limiting examples of biological samples include samples obtained from breast tissue, lymph nodes, and breast tumors. Biological samples also include any material derived from the body of a vertebrate animal, including, but not limited to, blood, cerebrospinal fluid, serum, plasma, urine, nipple aspirate, fine needle aspirate, tissue lavage such as ductal lavage, saliva, sputum, ascites fluid, liver, kidney, breast, bone, bone marrow, testes, brain, ovary, skin, lung, prostate, thyroid, pancreas, cervix, stomach, intestine, colorectal, brain, bladder, colon, nares, uterine, semen, lymph, vaginal pool, synovial fluid, spinal fluid, head and neck, nasopharynx tumors, amniotic fluid, breast milk, pulmonary sputum or surfactant, urine, fecal matter and other liquid samples of biologic origin.

The MIPs as described in the invention herein may be provided for use in a variety of media, sensors, devices, or products. For example, the MIPs of the present invention may be contained in a solution. As such, the solution can be sprayed onto an article to detect the target virus. In some embodiments, the solution may also contain an antiviral agent. The viruses may be detected on a variety of articles, such as environmental surfaces in hospitals, sports equipment, or medical devices. Some examples of MIP-based sensors are described in U.S. Pat. Nos. 5,587,273, 6,680,210, 6,833,274, 6,967,103, 6,749,811 and 6,461,873; the portions of which that describe MIP-based sensors are specifically incorporated herein by reference.

In hospitals, the transferal of viruses from environmental surfaces to patients is largely via hand contact with the surface. Although hand hygiene is important to minimize the impact of transfer, cleaning and disinfecting environmental surfaces as appropriate is fundamental in reducing their potential contribution to the incidence of healthcare-associated infections. Thus, the MIP products of the invention described herein include, but are not limited to, a hand-wipe, impregnated with a solution of MIPs designed to detect a virus, that could aid in insuring that hands of clinical personnel are free of the virus by changing color if the virus is present on their hands, a spray of the solution that could be used in high touch areas (e.g., bed railing, door knobs, computer keyboards, etc.). Such MIP products could be used to demonstrate the effectiveness of cleaning efforts. Such MIP products could also be valuable in outbreak investigations. For example, by being able to distinguish between various viruses, for example, HIV-1,HIV-1, HTLV-1 etc., it is possible to trace the path by which it is spread. Also, such MIP products can be used as an educational tool for training hospital staff.

The invention described herein also provides kits comprising MIPS as described above for specifically detecting, identifying and/or quantifying virus. Such kits include, but are not limited to, dipstick, lateral-flow, flow-through, and migratory devices with one or more MIPs attached to a mobile or immobile solid phase material such as latex beads, glass fibers, glass beads, cellulose strips or nitrocellulose membranes, as described in U.S. Pat. Nos. 3,802,842, 3,915,639, 4,059,407, 4,373,932, 4,689,309, 4,703,017; 4,743,560, 4,770,853 5,073,484, 5,075,078; 5,096,837, 5,229,073, 5,354,692, 7,109,042, WO 88/08534 and WO 08/007359, the portions of which that describe the construction and function of above-mentioned kits and devices are incorporated herein by reference. Dipstick devices, such as disclosed by Hochstrasser (U.S. Pat. No. 4,059,407) are designed to be immersed in a fluid biological sample and to a give a semi-quantitative estimation of the target in the fluid. Dipsticks are essentially lateral flow devices whose application method involves immersing the device in the liquid sample. Also of interest in the area of dipstick devices are U.S. Pat. Nos. 3,802,842, 3,915,639 and 4,689,309.

Lateral flow devices (see U.S. Pat. Nos. 5,075,078; 5,096,837; 5,354,692 and 5,229,073) generally comprise a porous matrix containing the relevant specific reagents, which is layered on a solid strip, such as plastic. Instead of vertically wicking the samples up the “dipstick,” the lateral flow format allows a sample to flow laterally across the porous, solid phase material by capillary action, across one or more reagents that interact with the target (if it is present in the sample). A visual signal (produced by colored beads, enzymatic reaction or other color-forming reactions) indicates the presence of the target.

In flow-through type devices, the applied test sample flows through a porous material, bringing the target in the sample in contact with the specific reagents contained in the porous material, eventually producing a visible signal on the porous material that provides an indication of the presence of target in the sample.

Visible detection of test results without the need to add external reagents is achieved in migration assay devices by incorporating reagents that have been coupled to colored labels (i.e., conjugates), thereby permitting visible detection of the assay results without addition of further substances. Such labels include, but are not limited to, gold sol particles, dye sol particles and dyed latex. In one embodiment, the diagnostic test devices of the invention described herein comprise two distinct pathways for the sample and the conjugated reagent.

One embodiment of the invention provides for a quantitative chromatographic test strip. The device consists of a strip that moves the sample solution by capillary action to zones in the strip containing the reagents that in the presence of the target produce a detectable signal.

Another embodiment of the invention discloses a chromatographic test strip comprising a solid support having two portions that permit capillary flow that is useful in a variety of immunoassays. The first portion includes a movable tracer and the second portion includes an immobilized binder capable of binding to the target.

Reagent-impregnated test strips have been used in various specific binding assays. The sample is applied to one portion of the test strip and migrates through the porous strip material, in some cases with the aid of an eluting solvent such as water. The sample advances into or through a detection zone where a specific binding reagent for the examined target is immobilized. The target present in the sample is then entrapped within the detection zone. The amount of bound target is determined usually by using labeled reagents incorporated in the test strip or applied subsequently. A variety of labels, such as radiolabels, chromophores, colored particles (gold, latex), enzymes, and fluorescent labels may be used in these assays. In most cases, the detecting binding agents are analyte-specific antibodies.

Further, in one embodiment, the invention is directed to a diagnostic device comprising a solid support capable of conveying a liquid sample therethrough, the sample being movable along or through the solid support in the path of liquid flow by capillary action. The support comprises: (a) a defined sample application area for applying the sample to the device and bringing it in contact with the solid support; (b) a defined MIP-conjugate zone downstream of the sample application area comprising a target-specific MIP fixed to the solid support on the flow path of the sample. The MIP has target-specific binding sites saturated with a releasable target analog:reporter conjugate in a dry state. The affinity of target to the binding sites of the target-specific MIP is greater than the affinity of the target analog:reporter conjugate to the binding sites of the target-specific MIP. The MIP, when contacted with a liquid sample containing the target, is capable of binding the target and displacing the target analog:reporter conjugate in an amount directly proportional to the concentration of the specific target, causing the displaced target analog:reporter conjugate to flow downstream in the path of liquid flow; (c) a defined results zone comprising a target analog:reporter conjugate binding element fixed to the solid support on the flow path of the sample downstream of the MIP-conjugate zone. The reporter conjugate binding element is capable of binding the target analog: reporter conjugate displaced from the MIP-conjugate zone when a liquid sample containing the target flows in the flow path zone for providing a detectable signal that indicates the presence or concentration of the target in a sample. Optionally, the solid support further comprises a reference zone for establishing a reference point in determining the presence or semi-quantification of an target in the tested sample, wherein the reference zone is not capable of capturing by specific binding any compound in said sample. The support may also optionally include a positive control zone comprising means for generating a positive control confirming the proper flow and binding of the target analog:reporter conjugate to the results zone to thereby determine that a test is working. Additionally, the support may optionally include an absorbent zone comprising a pad of absorbent material in fluid communication with the solid support when the pad and solid support are wet, the pad having sufficient porosity and capacity to absorb excess liquid.

In one embodiment, a sensor device as described herein comprises a molecularly imprinted polymer, containing a chelated lanthanide, capable of binding the target to be detected, and which has operatively associated therewith: a light source for generating excitation energy for the chelated lanthanide of the molecularly imprinted polymer, wherein at least a portion of the excitation energy is absorbed molecularly imprinted polymer; and a detector for detecting luminescent energy generated by the chelated lanthanide upon excitation.

It is to be understood that application of the teachings of the present invention to a specific problem or environment will be within the capability of one having ordinary skill in the art in light of teachings contained herein. The present invention is more fully illustrated by the following non-limiting examples.

### EXAMPLES

### Example 1

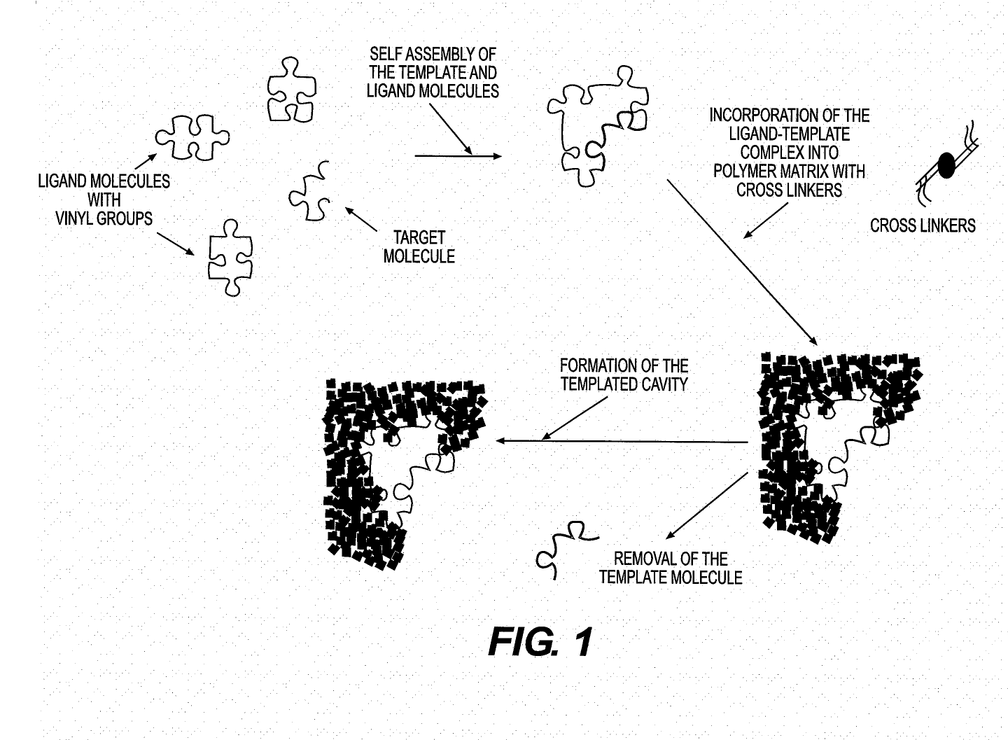

As illustrated in FIG. 1, MIPs of the invention described herein can be manufactured by generating a template of a portion of the target molecule, and polymerizing functional monomers in the presence of such template. The functional monomers can bind to active sites on the template molecule and then polymerize in the presence of excess of cross-linking agents. While the polymerization can be effected in the presence of the template molecules, subsequent removal of the latter can leave behind cavities that have the shape and an arrangement of the functional group that is complementary to that of the template molecules. Thus the resulting MIP can exhibit the ability to rebind the template molecule tightly and selectivity.

### Example 2

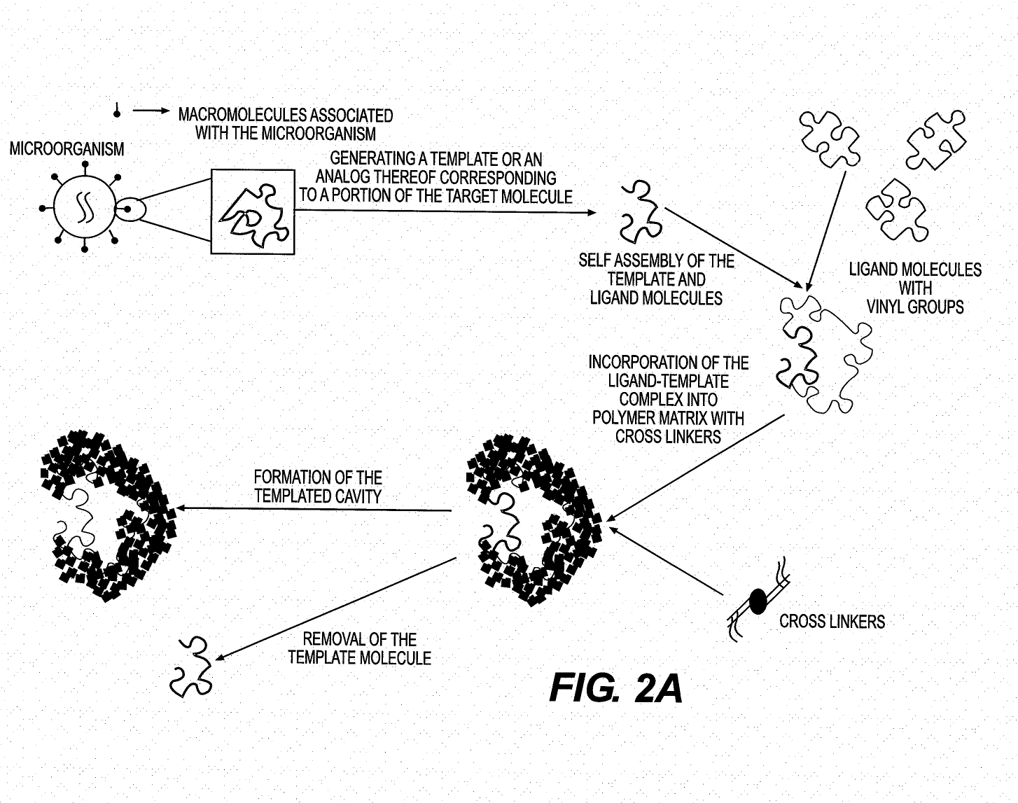

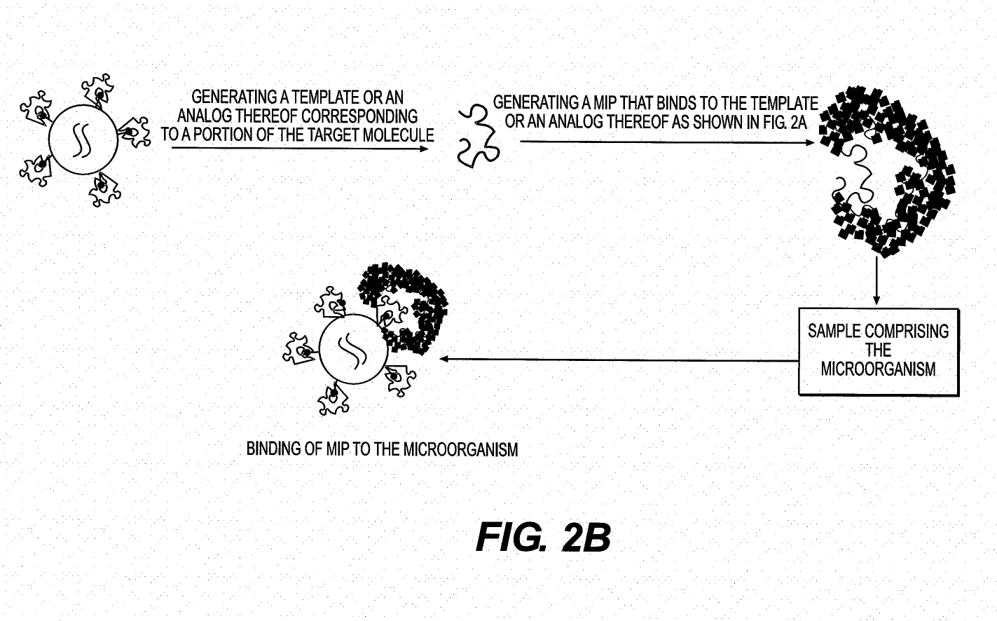

FIGS. 2A and 2B illustrate a schematic representation of detecting virus utilizing MIPs of the present invention. Upon identification of a unique macromolecule associated with the virus, a template of a portion of the macromolecule comprising one or more epitopes, can be generated. Functional monomers can be polymerized in the presence of the template molecule such that the monomers bind to active sites on the template molecule, which can further be polymerized in the presence of excess of cross-linking agents. Subsequent removal of the template molecule (FIG. 2A) can leave behind cavities that have the shape and an arrangement of the functional group that is complimentary to that of the portion of the macromolecule that is unique to the virus. The resulting imprinted polymer can thus exhibit the ability to bind the portion of the macromolecule associated with the virus tightly and selectively, and identify the virus (FIG. 2B).

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

Modifications may be made without departing from the basic spirit of the present invention. Accordingly, it will be appreciated by those skilled in the art that within the scope of the appended claims, the invention may be practiced other than has been specifically described herein

What is claimed is:

1. A molecularly imprinted polymer (MIP) capable of binding to all or a portion of a macromolecule associated with Human Immunodeficiency virus-1 (HIV-1).

2. The MIP of claim 1, wherein said macromolecule is gp160.

3. The MIP of claim 2, wherein the binding of gp160 to said MIP produces a detection signal.

4. The MIP of claim 3, wherein the binding of said portion of a macromolecule comprising amino acid sequences selected from the group consisting of SEQ ID NO:1 to SEQ ID NO:64, or fragments thereof produces a detection signal.

5. The MIP of claim 1, wherein said portion of a macromolecule is selected from the group consisting of SEQ ID NO:1 to SEQ ID NO:64, or fragments thereof.

6. The MIP of claim 1, wherein the MIP comprises a transduction element such that a measurable signal is produced in response to binding of HIV-1 to said MIP.

7. A method of detecting HIV-1 in a biological sample, comprising:
contacting said biological sample with a MIP capable of binding to all or a portion of a macromolecule associated with Human Immunodeficiency virus-1 (HIV-1).

8. The method of claim 7, wherein said macromolecule is gp160.

9. The method of claim 7, wherein said portion of a macromolecule is selected from the group consisting of SEQ ID NO:1 to SEQ ID NO:64, or fragments thereof.

10. The method of claim 7, wherein the MIP comprises a transduction element such that a measurable signal is produced in response to binding of all or a portion of a macromolecule associated with HIV-1 to said MIP.

11. The method of claim 10, wherein said portion of a macromolecule is selected from the group consisting of SEQ ID NO:1 to SEQ ID NO:64, or fragments thereof.


SEQUENCE LISTING (64 sequences)

SEQ ID NO: 1 - protein, 7 aa, Homo sapiens
CSGKLIC

SEQ ID NO: 2 - protein, 7 aa, Homo sapiens
GPGRAFY

SEQ ID NO: 3 - protein, 11 aa, Homo sapiens
RILAVERYLKD

SEQ ID NO: 4 - protein, 6 aa, Homo sapiens
ELDKWA

SEQ ID NO: 5 - protein, 7 aa, Homo sapiens
ELDKWAG

SEQ ID NO: 6 - protein, 8 aa, Homo sapiens
GPCGRAFY

SEQ ID NO: 7 - protein, 8 aa, Homo sapiens
ELLDKWAG

SEQ ID NO: 8 - protein, 12 aa, Homo sapiens
RIVALVERYLKD

SEQ ID NO: 9 - protein, 7 aa, Homo sapiens
GPGRAFY

SEQ ID NO: 10 - protein, 7 aa, Homo sapiens
ELDKWAS

SEQ ID NO: 11 - protein, 18 aa, Homo sapiens
RSKIRIQRGPGRAFVTIG

SEQ ID NO: 12 - protein, 6 aa, Homo sapiens
QRGPGR

SEQ ID NO: 13 - protein, 10 aa, Homo sapiens
CVAMKCSSTE

SEQ ID NO: 14 - protein, 10 aa, Homo sapiens
SSTGNNTTSK

SEQ ID NO: 15 - protein, 10 aa, Homo sapiens
STSTTTTTPT

SEQ ID NO: 16 - protein, 10 aa, Homo sapiens
DQEQEISEDT

SEQ ID NO: 17 - protein, 10 aa, Homo sapiens
PCARADNCSG

SEQ ID NO: 18 - protein, 10 aa, Homo sapiens
LGEEETINCQ

SEQ ID NO: 19 - protein, 10 aa, Homo sapiens
FNMTGLERDK

SEQ ID NO: 20 - protein, 15 aa, Homo sapiens
KKQYNETWYSKDVVC

SEQ ID NO: 21 - protein, 5 aa, Homo sapiens
ETNNS

SEQ ID NO: 22 - protein, 10 aa, Homo sapiens
TNQTQCYMNH

SEQ ID NO: 23 - protein, 10 aa, Homo sapiens
CKRPGNKIVK

SEQ ID NO: 24 - protein, 10 aa, Homo sapiens
QIMLMSGHVF

SEQ ID NO: 25 - protein, 10 aa, Homo sapiens
HSHYQPINKR

SEQ ID NO: 26 - protein, 6 aa, Homo sapiens
PRQAWC

SEQ ID NO: 27 - protein, 12 aa, HOMO SAPIENS
DDYQEITLNVTE

SEQ ID NO: 28 - protein, 9 aa, HOMO SAPIENS
SEDTPCARA

SEQ ID NO: 29 - protein, 8 aa, HOMO SAPIENS
GEETINCQ

SEQ ID NO: 30 - protein, 6 aa, HOMO SAPIENS
FNMTGL

SEQ ID NO: 31 - protein, 9 aa, HOMO SAPIENS
YSKDVVCET

SEQ ID NO: 32 - protein, 12 aa, HOMO SAPIENS
DTNYSGFAPNCS

SEQ ID NO: 33 - protein, 11 aa, Homo sapiens
LMSGHVFSHYQ

SEQ ID NO: 34 - protein, 10 aa, Homo sapiens
SGHVFHSHYQ

SEQ ID NO: 35 - protein, 12 aa, Homo sapiens
DDYQEITLNVTE

SEQ ID NO: 36 - protein, 11 aa, Homo sapiens
DDYQEPTLVTE

SEQ ID NO: 37 - protein, 12 aa, Homo sapiens
GDYSELALNVTE

SEQ ID NO: 38 - protein, 9 aa, Homo sapiens
NDTDPCIKT

SEQ ID NO: 39 - protein, 9 aa, Homo sapiens
NDTDPCIQL

SEQ ID NO: 40 - protein, 9 aa, Homo sapiens
NETSSCIAQ

SEQ ID NO: 41 - protein, 9 aa, Homo sapiens
GEEETINCQ

SEQ ID NO: 42 - protein, 9 aa, Homo sapiens
GEEEDINCE

SEQ ID NO: 43 - protein, 9 aa, Homo sapiens
REEDTDNCQ

SEQ ID NO: 44 - protein, 9 aa, Homo sapiens
EQEQMIGCK

SEQ ID NO: 45 - protein, 6 aa, Homo sapiens
FNMTGL

SEQ ID NO: 46 - protein, 6 aa, Homo sapiens
ENDTGL

SEQ ID NO: 47 - protein, 9 aa, Homo sapiens
YSKDVVCET

SEQ ID NO: 48 - protein, 9 aa, Homo sapiens
YSADVVCET

SEQ ID NO: 49 - protein, 9 aa, Homo sapiens
YSKDVVCES

SEQ ID NO: 50 - protein, 10 aa, Homo sapiens
YSTDLVVCET

SEQ ID NO: 51 - protein, 12 aa, Homo sapiens
DTNYSGFEPNCS

SEQ ID NO: 52 - protein, 12 aa, Homo sapiens
DTNYSGFMPKCS

SEQ ID NO: 53 - protein, 15 aa, Homo sapiens
INCTRPNNNTRKSIR

SEQ ID NO: 54 - protein, 15 aa, Homo sapiens
PNNNTRKSIRIQRGP

SEQ ID NO: 55 - protein, 15 aa, Homo sapiens
RKSIRIQRGPGRAFV

SEQ ID NO: 56 - protein, 14 aa, Homo sapiens
RKSIRIQRPGRAFV

SEQ ID NO: 57 - protein, 15 aa, Homo sapiens
IQRGPGRAFVTIGKI

SEQ ID NO: 58 - protein, 15 aa, Homo sapiens
GNMTQAHCNISRAKW

SEQ ID NO: 59 - protein, 15 aa, Homo sapiens
AHNCISRAKWNNTLK

SEQ ID NO: 60 - protein, 15 aa, Homo sapiens
GRAFVTIGKIGNMRQ

SEQ ID NO: 61 - protein, 15 aa, Homo sapiens
TIGKIGNMRQAHCNI

SEQ ID NO: 62 - protein, 15 aa, Homo sapiens
VKIEPLGVAPTKAKR

SEQ ID NO: 63 - protein, 15 aa, Homo sapiens
LGVAPTKAKRRVVQR

SEQ ID NO: 64 - protein, 15 aa, Homo sapiens
EVGKAMYAPPISHQI